(19) United States
(12) Patent Application Publication
(10) Pub. No.: US 2006/0088478 A1
(43) Pub. Date: Apr. 27, 2006

(54) Apparatus and method for applying cosmetic and/or therapeutic formulations

(76) Inventors: Neal Andrews, Citrus Heights, CA (US); Sandra Hayman, Harbor City, CA (US)

Correspondence Address: WOOD, NGO & EISENBERG, PLLC, 2121 Eisenhower Ave, Suite 200, Alexandria, VA, 22314, US

(21) Appl. No.: 11/258,271

(22) Filed: Oct. 26, 2005

Related U.S. Application Data
(60) Provisional application No. 60/621,692, filed on Oct. 26, 2004.

(51) Int. Cl. A61L 9/04; A61M 11/00

(52) U.S. Cl. 424/45; 128/200.23

(57) ABSTRACT

An apparatus, a method for manufacturing the apparatus, and a method for dispensing a formulation to a person or animal using the apparatus, wherein the apparatus includes: a dispensing container; an amount of formulation contained in the container; and a propellant contained in the container, wherein the propellant comprises between about 40% and about 100% oxygen.

### CROSS-REFERENCE TO RELATED APPLICATIONS

This application claims the benefit of priority from U.S. Provisional Patent Application Ser. No. 60/621,692, filed Oct. 26, 2004, the entire contents of which are incorporated herein by reference.

### STATEMENT REGARDING FEDERALLY SPONSORED RESEARCH OR DEVELOPMENT

Not Applicable.

1. Field of the Invention

This invention relates to an apparatus and method for applying cosmetic and/or therapeutic formulations.

2. Background of the Invention

A person's appearance and health are valuable assets and anything that can help maintain or improve these assets in an ethical and meaningful manner is of benefit to mankind. For example, beauty products including, but not limited to, skin treatment lotions are often used to maintain or otherwise beneficially condition a person's skin. The same principals apply to animals such as much loved family pets that sometimes require cosmetic or therapeutic topical treatment.

A person typically applies a skin treatment lotion or ointment by opening or squeezing a container such as a plastic bottle containing such lotion and applying the treatment directly to the skin usually by first transferring a desired amount of lotion from the container to one hand or finger and thence applying the lotion onto a desired area of skin. The step of first transferring an amount of lotion or ointment from the bottle to a finger or hand can be messy and wasteful. For example, a container such as a plastic squeeze bottle may spurt suddenly when compressed resulting in an uncontrolled amount of lotion coming out of the bottle causing an undesired mess.

In addition, not everyone can quickly and effortlessly squeeze or open a container. Some people suffer from arthritis and/or neurological disorders that make it difficult and/or uncomfortable to squeeze or otherwise access the contents of a container. Still other people would prefer a new way of applying lotion or ointment to their body. Thus, there is a strong need for an apparatus and/or method that can provide lotion or ointment in a more direct and controlled manner.

### BRIEF SUMMARY OF THE INVENTION

The invention is an apparatus and method for applying topical cosmetic and/or therapeutic formulations to a living thing such as a human or animal pet. In one embodiment of the invention, an amount of cosmetic and/or therapeutic formulation is added to an open container such as an aerosol-can. An aerosol valve component is attached to the can thus reversibly sealing the can, and gas containing oxygen is driven into the can under pressure through the valve component to provide a pressured container that contains a formulation and oxygen, wherein the oxygen (gas or liquid gas) acts as the propellant. An actuator in operable communication with the valve allows a person to operate the valve to release the formulation, wherein the oxygen forces the formulation out of the aerosol-can. The gas containing oxygen contains at least about 40% oxygen.

### BRIEF DESCRIPTION OF THE DRAWINGS

FIG. 1 shows a section view of a pressurized container at least partially filled with a cosmetic formulation, and a gas containing at least about 40% oxygen is present, the gas is under pressure to act as a propellant.

FIG. 2 shows a section view of a pressurized container according to the present invention.

FIG. 3 is a view of a pressurized container in combination with a straw according to the present invention.

It should be understood that the attached figures are used as examples only, and should not be regarded as limiting the scope of the present invention in any way.

### DETAILED DESCRIPTION OF THE INVENTION

This invention relates to an apparatus for dispensing a formulation and a method for applying cosmetic and/or therapeutic formulations to human and animal recipients. Target areas of the human or animal include the skin, hair, scalp, and the lining of body cavities. The present invention may be used, for example, to apply a topical application of soothing cream to moisturize dry skin.

The apparatus of the invention is generally denoted by the numeric label “100”.

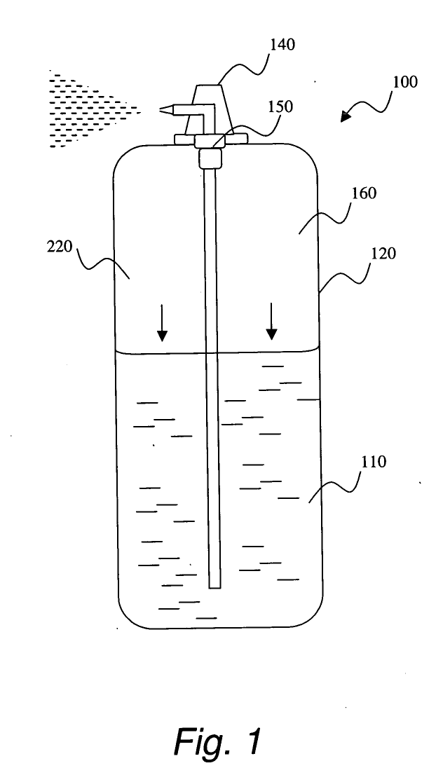

The apparatus 100 of the present invention might be manufactured as follows: a cosmetic formulation 110 is placed in a pressurized dispensing container 120 such as, but not limited to, an aerosol-can. A dispensing valve 150 is fitted to the top part of the container 120, and a suitable propellant 160, such as a gas propellant containing between about 40% and 100% oxygen (preferably between about 40% and about 95% oxygen, and still more preferably the propellant comprises about 95% oxygen), is added to the container 120 through the pressure valve to provide an aerosol-can filled with propellant 160 under pressure and, in this example, a cosmetic formulation 110 (see FIG. 1). The cosmetic formulation 110 could be any cosmetic formulation, such as a hair-coloring agent. An actuator, such as a nozzle 140, controls the pressure valve. It should be understood that the propellant 160 can be found in the formulation 110 or 180 such that a gap may not exist above the formulation inside the container 120. Thus, while a gap 220 is shown in FIGS. 1 through 3, this should not be construed to mean a gap 220 always exists above the formulation 110 and/or 180, alone or in combination. A gap 220 may not exist at all, or a gap 220 may develop as the apparatus 100 is used to apply formulation 110 or 180.

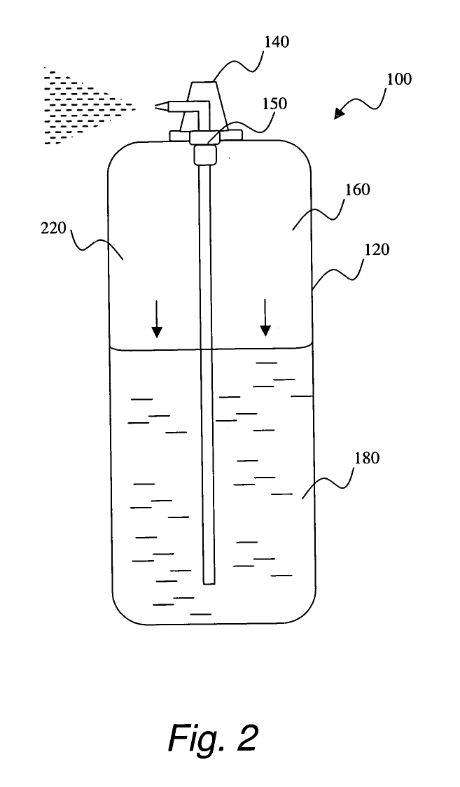

The apparatus 100 of the present invention might be manufactured as follows: a therapeutic formulation 180 is placed in a pressurized dispensing container 120, such as, but not limited to, an aerosol-can. A dispensing valve 150 is fitted to the top part of the container 120, and a suitable propellant 160, such as a gas propellant containing between about 40% and 100% oxygen (preferably between about 40% and about 95% oxygen and still more preferably the propellant comprises about 95% oxygen), added to the container 120 through the pressure valve to provide an aerosol-can filled with propellant 160 under pressure and, in this example, a therapeutic formulation 180 (see FIG. 2). The therapeutic formulation 180 could be any suitable therapeutic formulation, such as a therapeutic skin cream. An actuator such as a nozzle 140 controls the dispensing valve. A non-limiting example of a dispensing valve is described in U.S. Pat. No. 3,225,967 issued to Heimgartner (the Heimgartner dispensing valve is referred to as part number 4). The Heimgartner '967 patent is incorporated by reference herein in its entirety.

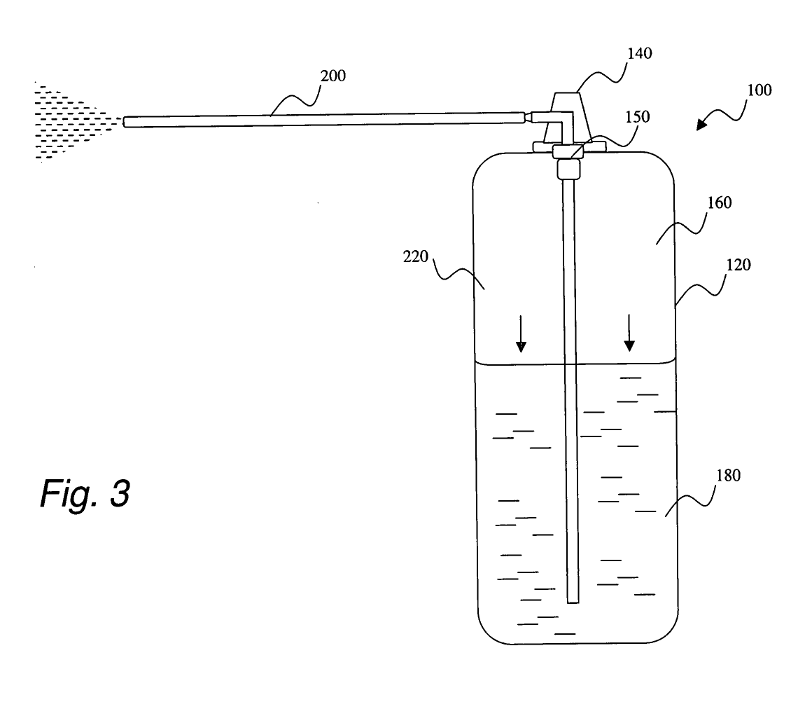

Referring to FIG. 3, an elongated member with open opposite ends and a hollow bore therebetween, such as a straw 200, can be attached or connected to the valve 150 to enable the container 120 to apply cosmetic 110 and/or therapeutic 180 formulations to areas of the body such as, but not limited to, awkward and/or hard to reach places or where the user (not shown) wants to apply a formulation to a specific area of the body thereby minimizing waste. The manner of applying a formulation of interest is in the form of a spray or foam or liquid. Specifically, the formulation 110 or 180 can be outputted from the device 100 in the form of a spray or foam or liquid.

The exact form of pressurized container 120 can vary. Pressurized containers for delivering such formulations as shaving foam are well known in the art. But containers for delivering cosmetic and/or therapeutic formulations, wherein the propellant is oxygen are believed by the Applicants to be previously unknown in the art of delivering such formulations. A non-limiting example of a pressurized dispensing container is described in U.S. Pat. No. 5,044,524 issued to Pistek, the Pistek '524 patent is incorporated by reference herein in its entirety.

In a preferred, but non-limiting embodiment, oxygen acts as the principal propellant for driving the cosmetic and/or therapeutic formulation out of the container (such as a pressurized container 120). The valve 150 can be opened in any conventional way such as by pressing the nozzle 140. For example, the valve 150 is preferably held in a closed position until opened by human intervention, e.g., by a person's finger pressing on the nozzle 140 (commonly referred to as “the button”).

Though the present invention has particular application with regard to any person requiring or desiring the topical application of a cosmetic and/or therapeutic formulation, the invention is also directed to the same with regard to an animal; e.g., an owner that desires their much loved family pet to receive a topical treatment with an appropriate formulation such as a soothing cream for application to a sore or dry skin area on the animal pet. Thus, the present invention explicitly does not exclude any live animal or human that can benefit from receiving topical applications as described herein.

It should be understood that the terms “cosmetic formulation”, “cosmetic and/or therapeutic formulations” and “therapeutic formulations” are intended to cover any useful composition that can be applied topically to the body such as, but not limited to: hair, scalp, and skin treatment compositions as disclosed in U.S. Pat. No. 4,201,235, issued May 6, 1980 to Ciavatta. The Ciavatta '235 patent is hereby incorporated by reference in its entirety. Other non-limiting examples of cosmetic and/or therapeutic formulations to which the present invention speaks include those listed in Table 1. All patents listed in Table 1 are incorporated herein by reference in their entirety.

An amount of cosmetic and/or therapeutic formulation is added to an open container, but minus the valve apparatus. The valve apparatus 150 is attached to the container 120 thus reversibly sealing the container 120, and oxygen is driven into the container 120 under pressure through the valve apparatus 150 to provide a pressured container that contains a formulation and oxygen propellant. The term “reversibly sealing” simply acknowledges that the valve used to seal the container 120 can be opened by an actuator, for example, by a person's finger applying pressure to an actuator in operable communication with the valve.

Since oxygen forms part of the air we breath, very little or no harm is done to the environment when oxygen is used as a propellant in place of the typical organic based solvent often used as propellants in traditional aerosol-cans. The Applicants are unaware of an aerosol-can that delivers cosmetic and/or therapeutic formulation using oxygen as the propellant.

TABLE 1*

| | Inventor(s); | |
|---|---|---|
| U.S. Pat. No. | issue date | Description of Cosmetic formulations |
| 3,968,218 | Bouillon et | Cosmetic composition containing a hydroxylated amino thioether for |
| | al.; | application to the hair and skin. |
| | 07/06/1976 | |
| 4,186,188 | Gumprecht; | Cosmetic formulations containing polypeptides. |
| | 01/29/1980 | |
| 4,201,235 | Ciavatta; | Compositions and methods for reducing the risk of sunlight and ultraviolet |
| | 05/06/1980 | induced skin cancer. Specifically compositions comprising mixtures of |
| | | certain amino acids and vitamins particularly useful in the formulation of |
| | | topically applicable cosmetic compositions. The topical application of the |
| | | compositions acts to improve the general complexion of the skin and to |
| | | invigorate and revitalize the hair and scalp. Separate formulations are |
| | | provided for each use. |
| 4,906,457 | Ryan; | Compositions and methods for reducing the risk of sunlight and ultraviolet |
| | 03/06/1990 | induced skin cancer; compositions include at least one effective protease |
| | | inhibitor. |
| 5,030,629 | Rajadhyaksha; | The Rajadhyaksha '629 patent describes cosmetic formulations comprising |
| | 07/09/1991 | heterocyclic compounds containing two heteroatoms as membrane |
| | | penetration enhancers. |
| 5,254,331 | Mausner; | The Mausner '331 patent describes a skin cream composition comprising |
| | 10/19/1993 | water, and emulsified and dispersed in the water: (1) a protein complex |
| | | comprising serum proteins and hydrolyzed animal proteins; (2) a protein- |
| | | amino acid-vitamin-nucleotide complex comprising proplylene glycol, |
| | | serum proteins, niacinamide, water, adenosine phosphate, and arginine; |
| | | and (3) dimethylsilanoyl hyaluronate complex, and the dimethylsilanoyl |
| | | hyaluronate are each present in cosmetically effective quantities. |
| | | Preferably, the skin cream composition further comprises a highly stable |
| | | micellar complex. |
| 5,603,926 | Matsumoto | The Matsumoto et al. '926 patent describes a cosmetic composition |
| | et al.; | comprising cationic polymer thickener prepared in a nonaqueous system in |
| | 02/18/1997 | the absence of a surface active agent by polymerizing monomer |
| | | composition containing 15 to 90% by weight of at least one of acrylic |
| | | monomer having amino group and methacrylic monomer having amino |
| | | group, 0 to 80% by weight of vinyl monomer, 1 to 60% by weight of |
| | | monomer having at least one of acryloyl group and methacryloyl group |
| | | and 0.1 to 25% by weight of crosslinkable vinyl monomer. This cosmetic |
| | | composition imparts refreshing feeling and little skin irritation and can be |
| | | suitably used as hair cream, hair lotion and the like. |
| 5,750,092 | Meyer et al.; | The Meyer et al. '092 describes a sunless tanning composition comprising |
| | 05/12/1998 | dihydroxyacetone and a fluid formulation comprising a secondary |
| | | polyamine, wherein the polyamine formulation also contains a color |
| | | modifier which is: an amino acid; a substituted ethylenediamine; and |
| | | mixtures of any two or more of the foregoing. |
| 5,908,836 | Bar-Shalom | The Bar-Shalom et al. '836 patent describes a medicament for topical |
| | et al.; | application to the skin or to any non-gastrointestinal, non-oral mucosal |
| | 06/01/1999 | surface of an animal or a human, including the lining of body cavities, or |
| | | for injection into tissue, including joints, or implantation into surgical |
| | | wounds or a body cavity of an animal or a human, for the prophylaxis or |
| | | treatment of any manifestation of inflammation or infection, for the |
| | | prophylaxis or treatment of non-bladder premalignant or malignant |
| | | disorders, for the prophylaxis or treatment of irritation or burns of the skin, |
| | | connective tissue, or non-oral mucosa, or for the prophylaxis or treatment |
| | | of skin, connective tissue, or mucosal aging, or for the preparation of a |
| | | medicament for systemic injection for the treatment or prophylaxis of |
| | | infectious, malignant or allergic/immune disorders. The sulphated |
| | | saccharide, e.g. sucrose octasulphate, may be in the form of a complex or a |
| | | salt with an alkali or alkaline earth metal (e.g. Na, K, Ca, Mg or Ba) or Al, |
| | | Zn, Cu, Zr, Ti, Bi, Mn or Os, or an organic base (e.g. an amino acid). The |
| | | medicament may be a powder, paste, ointment, lotion, gel, cream, salve, |
| | | emulsion, solution, suspension, spray, sponge, strip, plaster, pad, dressing |
| | | or ostomy plate. |
| 6,309,656 | Pugliese, | The Pugliese et al. '656 patent describes a hair and scalp formulation |
| | et al.; | based on a peroxidase enzyme and synergistic yeast extract therefore. It |
| | 10/30/2001 | entails a chemically-inert carrier material, a germicidal/preservative agent, |
| | | and a metal chelating agent. A skin protective formulation based on |
| | | peroxidase enzyme and yeast extract. It entails a polymer carrier, a fatty |
| | | acid emollient, a co-emulsifier of an amino acid complex essential to a |
| | | fatty acid; an emulsifying agent; a skin healing agent, and a germicidal |
| | | surfactant. An aqueous formulation adapted for topical hair rinsing |
| | | consisting of a peroxidase enzyme, a yeast extract adapted to enhance the |
| | | activity of the enzyme and a cationic hair conditioning agent selected from |
| | | water-soluble thickening resins. |

*Examples of non-limiting cosmetic and/or therapeutic formulations suitable for applying to a person's body according to the present invention wherein the cosmetic formulation is applied topically to the body from an aerosol-can and further wherein the principal propellant is oxygen. While other propellants can be used in combination with oxygen, oxygen is the preferred propellant.

### WORKING EXAMPLE #1

An open aerosol-can is filled with about 1 to 8 oz of a skin treatment formulation known as Amino-Plex® (supplied by biO₂ Cosmeceuticals of Harbor City, Calif., USA) is added to an open aerosol-can. A suitable pressure valve is attached to the can to seal the can and oxygen propellant is delivered into the can under pressure through the valve thus providing a pressurized can filled with Amino-Plex® product and oxygen, wherein the oxygen is under pressure and acts as the propellant. The Amino-Plex® formulation comprises amino acids, trace minerals, electrolytes, nucleotides, nucleosides (RNA/DNA), glycosamingoglycans, glycolipids, and micellized Vitamin E.

The Amino-Plex® formulation is ejected out of the aerosol-can in response to a person applying pressure to an actuator in operable communication with the valve such that the valve is opened in response to a finger applying pressure thereon to release the skin treatment formulation out of the can.

It will be understood by persons of ordinary skill in the art of consumer pressured containers such as, but not limited to, aerosol-cans, that the contents thereof may be release as foam and/or as an aerosol spray depending on the dimensions of the valve orifice and the pressure applied to the contents in the aerosol-can which in turn is a function of the amount of propellant (here oxygen) pushed through the valve into the aerosol-can. Thus, the skin treatment formulation may be released as foam much like shaving foam or as a spray.

### WORKING EXAMPLE #2

1.25 oz of a skin treatment formulation known as Amino-Plex® (supplied by biO₂ Cosmeceuticals of Harbor City, Calif., USA) is added to an open aerosol-can. A suitable pressure valve is attached to the can to reversibly seal the can and oxygen propellant is delivered into the can under pressure through the valve thus providing a pressurized can filled with Amino-Plex® product and oxygen, wherein the oxygen is under pressure and acts as the propellant.

The terms “applying a formulation” and “dispensing a formulation” are regarded as equivalent terms.

It is to be understood that the present invention is not limited to the embodiments described above or as shown in the attached figures, but encompasses any and all embodiments within the scope of the attached claims.

We claim:

1. An apparatus for applying a formulation to a person or animal, said apparatus comprising:
a dispensing container;
an amount of formulation contained in said container; and
a propellant contained in said container, wherein said propellant comprises between about 40% and about 100% oxygen.

2. The apparatus according to claim 1, wherein said propellant comprises between about 40% and about 95% oxygen.

3. The apparatus according to claim 1, wherein said propellant comprises about 95% oxygen.

4. The apparatus according to claim 1, wherein said formulation is a therapeutic formulation.

5. The apparatus according to claim 1, wherein said formulation comprises Amino-Plex®.

6. The apparatus according to claim 1, wherein said formulation is a cosmetic formulation.

7. The apparatus according to claim 1, wherein said formulation is any composition described in Table 1.

8. A method for manufacturing a formulation dispensing apparatus, the method comprising the steps:
adding a formulation 110 or 180 to a container 120;
fitting a dispensing valve 150 to the top part of the container 120;
adding a propellant to the container, wherein the propellant is added to the container through the valve, wherein the propellant contains between about 80% and 100% oxygen; and
an actuator is fitted to the apparatus such that the actuator is operably connected to the valve.

9. The method according to claim 8, wherein the propellant is about 100% oxygen.

10. The method according to claim 8, wherein the formulation is a cosmetic formulation.

11. The method according to claim 8, wherein the formulation is a therapeutic formulation.

12. The method according to claim 8, wherein the formulation is any composition described in Table 1.

13. A method for dispensing a cosmetic or therapeutic formulation, the method comprising the step of: dispensing a formulation from a container, whereby the act of dispensing the formulation is driven by a propellant, wherein the propellant comprises between about 80% and 100% oxygen.
